(19) United States
(12) Patent Application Publication
(10) Pub. No.: US 2006/0277615 A1
(43) Pub. Date: Dec. 7, 2006

(54) Method of determining xenograft responses

(76) Inventors: Charles Roland Wolf, Dundee (GB); Kenneth Brown, Dundee (GB)

Correspondence Address: MYERS BIGEL SIBLEY & SAJOVEC, PO BOX 37428, RALEIGH, NC, 27627, US

(21) Appl. No.: 10/551,861

(22) PCT Filed: Apr. 8, 2004
(86) PCT No.: PCT/GB04/01543

§ 371 Date: Sep. 30, 2005

(30) Foreign Application Priority Data
Apr. 9, 2003 (GB) 0308150.2

(51) Int. Cl. A01K 67/027; C12Q 1/68

(52) U.S. Cl. 800/14; 800/18; 435/6

(57) ABSTRACT

The present invention provides a method of modifying or engineering cells that are intended for introduction into a host as a xenograft, the modification or engineering of the cells comprising inclusion of at least one suitable reporter system. Inclusion of a reporter system permits monitoring of the xenograft and determination of xenograft parameters of interest through appropriate and convenient measurement systems.

The present invention relates to a method of genetically modifying or engineering cells or tissue and introducing said cells or tissue into a non-human animal, especially but not exclusively, as a xenograft. The genetic modification(s) made to the xenograft cells or tissue are such as to allow reporting of cell physiological processes within the cells implanted into the host animal, for the purpose, especially but not exclusively, of monitoring cell physiological processes within the xenograft and/or monitoring the effects of drugs or other therapeutic interventions on the xenograft. The present invention further includes products comprising genetically engineered cells and/or tissues comprising such genetically engineered cells and uses thereof.

### BACKGROUND TO THE INVENTION

Experimental xenografts in animals are an essential tool in cancer research and in studying the efficacy of anti-cancer drugs or other therapeutic procedures. Experimental xenografts also have potential applications more generally in the study of the mode of action of drugs or other chemical compounds or biological substances or agents in biological systems. For example, it is known from the prior art in U.S. Pat. No. 6,107,540 and U.S. Pat. No. 6,365,797 to use human prostate cell lines to generate a cancer xenograft in immuno-compromised SCID mice and to monitor disease progression and effects of therapeutics on the xenograft tumour. However, the information that can be gained from prior art experimental approaches is limited. This is due to the few informative parameters of xenograft physiology that can be measured sufficiently or effectively. In addition, there is the problem that the xenograft itself is only accessible by invasive or cumbersome methods, which typically necessitates culling of the animal. Accordingly, xenograft measurements may only be made at a limited number of specific time points. A yet further problem resides in quantitative assessment of xenograft cell proliferation status. Typically, measurement of xenograft size is used as the indicator however such measurements are prone to inaccuracies and are not capable of picking up subtle changes that may occur in progression or regression of a xenograft tumour.

An improved method of determining the quantity and quality of information that can be generated from the introduction of a xenograft into a host, including the ability to detect the very earliest stages of a response, based on knowledge of the mechanisms involved, before the response would be evident using existing methods, would offer immediate advantage to the prior art. Such a method would not only benefit the understanding of physiological processes of xenografts in a host but would also provide an improvement to the determination of efficacy of candidate therapeutic agents in xenograft animal models and their effect on physiological processes.

### STATEMENT OF THE INVENTION

The present invention resides in the modification or engineering of cells that are intended for introduction into a host as a xenograft, the modification or engineering of the cells comprising inclusion of at least one suitable reporter system. Inclusion of a reporter system advantageously allows for the determination of xenograft parameters of interest through appropriate and convenient measurement systems. Accordingly, many of the problems associated with the prior art can be mitigated and the information from experimental xenografts can be expanded.

According to a first aspect of the invention there is provided a method of monitoring progression of a xenograft in an animal model comprising:
(i) genetically modifying or engineering a cell so as to incorporate at least one reporter molecule and/or reporter gene into said cell either before or after implantation of said cell into an animal;
(ii) implanting said modified cell into said animal model and allowing a xenograft to grow for a sufficient period of time; and
(iii) measuring at least one parameter of a selected biochemical/physiological response associated with the reporter molecule or reporter gene.

Preferably, there is a plurality of genetically modified or engineered cells and more preferably the cells are human or non-human in origin and may be in the form of a primary isolate derived from, for example a tumour or they may be in the form of an immortalised or established cell line.

Preferably, the modified cells that are implanted into the animal host are selected from the group comprising cells derived from a tumour for example and without limitation a liver, brain, gut, adrenal, kidney, skin or any other organ or tissue which it is desired to xenograft. The cells may be prepared directly from such tumours or they may be an established cell line, for example and without limitation a cell line selected from the list of tumour cell lines listed at http://dtp.nci.nih.gov/branches/btb/tumor-catalog.pdf.

It will be appreciated that the xenograft cells of the present invention are not limited to tumour cells, they may be for example embryonic stem cells or they may be derived from any type of living cell of mammalian or non-mammalian origin. For example, bacterial cells that produce a suitable reporter response may be employed to detect certain parameters in the host animal.

The cells which have been transiently or stably modified or engineered so as to incorporate at least one reporter molecule or reporter gene into them will hereinafter be conveniently referred to as the “reporter cell/system”.

Throughout the description and claims of this specification, the words “comprise” and “contain” and variations of the words, for example “comprising” and “comprised”, mean “including but not limited to”, and are not intended to (and do not) exclude other components, integers, moieties, additives or steps.

Reference herein to a “reporter molecule” is intended to include chemical moieties used for labelling a nucleic acid or amino acid sequence. Preferably, they include, but are not limited to, proteins, antigens, enzymes, enzyme substrates, fluorescent, chemi-luminescent, chromogenic agents or radionuclides. Reporter molecules associate with, establish the presence of, and may allow quantification of a particular nucleic or amino acid sequence.

Reference herein to “reporter genes” are intended to include nucleic acids and fragments thereof encoding a functional protein. The reporter genes referred to in the present invention can “report” many different properties and events, for example apart from normal physiological processes they can report the strength of promoters, whether native or modified for reverse genetics studies; the efficiency of gene delivery systems; the intracellular fate of a gene product; a result of protein traffic; interaction of two proteins in the two-hybrid system or of a protein and a nucleic acid in the one-hybrid system; the efficiency of translation initiation signals; the success of molecular cloning efforts; and effects of exogenous agents on physiological processes.

Reporter genes are nucleic acid sequences encoding directly or indirectly assayable proteins. They are used to replace other coding regions whose protein products are unsuitable or not amenable to the assay envisaged. Suitable reporter genes that are konown in the art and may be used in the method of the present invention are selected from those genes encoding proteins including but not limited to: chloramphenicol-acetyltransferase, β-galactosidase, β-glucuronidase, luciferase, beta-galactosidase, green fluorescent protein, secreted alkaline phosphatase (SEAP), major urinary protein (MUP) or human chorionic gonadotrophin (hCG). It will be understood that the above list of suitable reporter genes is not exhaustive or exclusive and is not intended to limit the scope of the application. The skilled artisan may select another reporter system which will equally be applicable to the method of the present invention.

It will be appreciated that reporter genes can be attached to other sequences so that only the reporter protein is made or so that the reporter protein is fused to another protein (fusion protein).

Reference herein to a “reporter agent” is intended to include a protease or kinase or protein or RNA or any other biochemical moiety that effects changes in cell protein or mRNA stabilisation.

Preferably, the animal model is a rodent and more preferably is a mouse or rat of wild type or of a specifically selected genetic background, for instance one in which drug metabolism characteristics are modified.

Preferably, the animal model may have more than one different population of reporter cells/system implanted therein. The method of the present invention comprises using genetically engineered cells of human or non-human origin so as to become a reporter system by the incorporation of one or more reporter molecules or reporter genes or transgenes into the selected cells preferably in a manner permitting replication of the incorporated reporter genes with replication of the host cell genome. Preferably, the reporter molecule or transgene is suitable for the purpose of allowing convenient monitoring of cell physiological processes in vivo when the cells are implanted into a non-human animal. “Read-out” or information from the reporter cells or system may be in the form of qualitative or quantitative data and may involve invasive or non-invasive procedures in order to ascertain this data. Accordingly, one method of the present invention conveniently provides an animal model wherein multiple measurements may be made over a protracted period of time.

Preferably, the method of the invention further includes allowing the xenograft to proliferate as a xenograft tumour.

Preferably, the reporter cells/system may be introduced into an animal host either as individual cells suspended in suitable medium or as tissue fragments. The reporter cells/system may grow in the host animal either systemically, or as a xenograft tumour at the site of implantation, possibly but not necessarily as a tumour with or without metastatic tumours at secondary sites.

Preferably, the host animal is immuno-suppressed by means of administration of appropriate immuno-suppressant agents or is of an immuno-compromised strain, for example and without limitation SCID. Alternatively, the reporter system may be grown in an immunologically intact animal where the reporter cells are syngeneic with the host animal.

Preferably, the reporter cells/system is/are genetically engineered to express a transgene or multiple transgenes. The reporter cells/system may be already expressing the transgene(s) at the time of implantation or may be transfected in vivo with a transgene in a specifically targeted manner, for example and without limitation, by means of a viral vector. Accordingly, it will be understood that the method of the present invention conveniently allows for transfection of cells prior to implantation or after implantation of the cells into the host animal.

Preferably, the reporter cell/system transgenes may comprise elements that allow measurement of relevant biochemical parameters in response to changes in cell physiology occurring during reporter cell proliferation or brought about by toxicological or pharmacological effects of administered compounds or biological substances.

Preferably, cell physiological processes that may be monitored by means of a suitable reporter molecules or transgenes include, for example and without limitation:
a) Parameters of xenograft growth or differentiation or death, such as for instance reporter cell numbers, cell cycle modulation or mitotic fraction, cell differentiation, angiogenesis, hypoxia, cell death or lysis (for instance by apoptosis, necrosis);
b) Mechanisms of toxicity, such as for instance oxidative stress, DNA damage, mitochondrial function, membrane perturbation, GSH depletion, receptor-mediated toxicity, enzyme inhibition, cofactor availability, pH or osmotic change, perturbation of calcium homeostasis, cell differentiation, protein turnover, ubiquitination, protein misfolding;
c) Mechanisms of drug action, such as for instance effects on intracellular signalling pathways, receptor interactions, effects on gene transcription, translation or protein stability, hormone or growth factor receptor modulation; peroxisome proliferator-activated receptor modulation, intracellular signal transduction pathways such as for instance MAP kinase or phosphatase signalling, p53 signalling, ras signalling;
d) Induction of drug resistance mechanisms, drug delivery or drug bystander effects.

Reporter cells may preferably be genetically engineered to facilitate determination of the above processes through incorporation of at least one transgene whose expression products permit convenient determination of relevant parameters.

Suitable transgenes preferably comprise a naturally occurring or artificial promoter sequence driving expression of a gene resulting in production of a reporter protein.

Preferably, the reporter expression products are detectable transcriptionally or post-transcriptionally.

In one embodiment of the invention (transcriptional reporting), the ability of the promoter to drive gene transcription is dependent either positively or negatively on the relevant parameters so that direct sensing of the gene products provides a read out of the relevant parameters.

In another embodiment of the invention (post-transcriptional reporting), the promoter is constitutively active or inducible by factors independent of the parameters to be determined, but the gene products have effects on or are affected by processes within the reporter cell in a manner that facilitates readout of the relevant parameters. Examples of such a method of obtaining read out include, for instance: a gene transcript or protein whose stability is positively or negatively dependent on the relevant parameters; a protein whose post-translational modification state, for instance its degree of phosphorylation, or whose subcellular localization or whose secretion from the expressing cell is dependent on the relevant parameters, or a protein with enzymatic activity catalysing modifications of either endogenous gene transcripts or proteins or products of other transgenes in the same cell such that those modifications provide a measurable read out.

It is envisaged that in the embodiment using transcriptional reporting, it could be mediated, for example, through suitable gene promoters, for example and without limitation the vascular endothelial growth factor (VEGF) promoter to detect hypoxia; the inducible nitric oxide synthetase (iNOS) promoter or the haemoxygenase-1 (HO-1) promoter or the cyclo-oxygenase-2 (COX-2) promoter to detect oxidative stress; the tissue transglutaminase promoter or the Peg3/pwl promoter to detect apoptosis, the 14-3-3 protein promoter or the GADD153 promoter to detect DNA damage. It will be appreciated that the foregoing list of suitable gene promoters is not intended to be limiting in the method of the present invention. The list of promoters is neither exhaustive nor exclusive and the skilled artisan may select any promoter that is capable of detecting a cellular physiological parameter that it is desired to monitor or measure.

In the embodiment employing post-transcriptional reporting, it could be effected through for example generation of a protein that can effect protein modifications, for example as a consequence of protease activity that result in translocation of a cytoplasmic protein to the nucleus or from membrane-bound form to secreted form or through protein cleavage that could for instance result in activation of a proenzyme or transcription factor or deactivation of an active enzyme or transcription factor or in secretion into the blood or excretion into urine (e.g. Alanine aminotransferase). Post-transcriptional reporting could also involve, for instance, production of a protein or RNA that effects changes in the stabilisation of a protein or mRNA.

Preferably, the reporter cells/system may comprise genetic elements to facilitate detection of responses to relevant cell physiological parameters or intracellular signalling pathway activation. For instance, products of transgene expression may be chosen to allow convenient non-invasive assays in excreted body products, for instance in urine (for instance, human chorionic gonadotrophin, hCG), faeces, breath or saliva. Alternatively, products of transgene expression may be chosen to allow assay by non-invasive procedures, for instance by bioluminescence measurement, by blood pressure measurement, by transcutaneous oxygen tension measurement, by nuclear magnetic resonance measurement or by positron emission tomographic measurement of, for instance, glucose utilisation. Alternatively, products of transgene expression may be chosen to allow convenient invasive assays, for instance in blood (for instance secreted alkaline phosphatase, SEAP) or in xenograft tissue (for instance β-galactosidase or by “real-time” PCR of RNA from cells).

Transgene reporter products that are measured in blood, tissues or body products may preferably be assayed by, for instance, immunoassay (for instance radioimmunoassay or enzyme-linked immunoassay, ELISA) or by enzymatic assay, or by colorimetric assay or by chromatographic assay (for instance HPLC) or by mass spectrometric assay or by nuclear magnetic resonance spectroscopy.

In another embodiment of the invention the reporter cells/system may incorporate multiple transgenes that could, for example, permit multiple forms of “read-out” of individual parameters from invasive and non-invasive intervention or simultaneous measurement of multiple parameters of interest, or to control for measured parameters being secondary to other parameters for example, cell proliferation that is secondary to angiogenesis.

Alternatively, reporter protein products of transgene expression under the control of different promoters may be distinguished by immunoassay by, for instance, incorporating into coding sequences of the transgene, coding sequences for clearly distinguishable epitope tags.

Particular advantages of the method of the present invention as described in step (iii) of the method is in providing methods of:
a) Making accurate measurement of reporter cell proliferation, particularly during xenograft growth or differentiation or death or in response to treatments;
b) Determining the mechanism of differentiation or death where reporter cell differentiation or death occurs;
c) Monitoring processes in secondary metastatic tumours to establish whether these differ in their responses from the primary reporter cell xenograft;
d) Making non-invasive measurements of parameters related to biochemical processes in the reporter cells;
e) Identifying drug-resistant cell populations, for instance arising from differential toxicity of a drug to dividing as compared to non-dividing cells or to hypoxic as opposed to normoxic cells;
f) Determining the effects of genetic background on tumour cell growth or on response to treatments, for instance in cells expressing and not expressing p53;
g) Making dynamic measurements using reporters of short half lives or that are excreted; and
h) Determining or confirming the targets of drug action in vivo; and
i) Measuring drug bystander effects; and identifying promoter elements involved in gene regulation; and
j) Determining intracellular drug concentrations and thereby those cells that take up a drug.

According to a further aspect of the invention there is provided a product comprising a genetically modified or engineered cell, the modification or engineering of the cell being such that the cell comprises at least one reporter molecule or reporter gene.

Preferably, the product further comprises any one or more of the features hereinbefore described.

According to a yet further aspect of the invention there is provided an artificial gene construct comprising a promoter region of the SFN gene and human chorionic gonadotrophin (hCG) such that expression of hCG is controlled by the SFN promoter.

Preferably, the 5′-regulatory promoter region of the SFN gene is linked to the excretable reporter human chorionic gonadotrophin (hCG).

In one embodiment of the invention the 3′ end is linked to Amp and the hCG includes an epitope tag such as myc, however it will be appreciated by those skilled in the art that other suitable variations to the 3′ end may be made.

According to a yet further aspect of the invention there is provided a human tumour-derived cell line containing this construct as hereinbefore described.

According to a yet further aspect of the invention there is provided an animal model containing a human tumour-derived cell line containing this construct as hereinbefore described.

According to a yet further aspect of the invention there is provided use of the method or product of the present invention in any one or more of the following examples:
(a) measuring reporter cell proliferation, particularly but not exclusively during xenograft growth or differentiation or death or in response to treatments;
(b) determining the mechanism of differentiation or death where reporter cell differentiation or death occurs;
(c) monitoring processes in secondary metastatic tumours where these may differ in their responses from the primary reporter cell xenograft;
(d) making non-invasive measurements of parameters related to biochemical processes in the reporter cell/system;
(e) identifying drug-resistant cell populations, for example arising from differential toxicity of a drug to dividing as compared to non-dividing cells or to hypoxic as opposed to normoxic cells;
(f) determining the effects of genetic background on tumour cell growth or on response to treatments, for instance in cells expressing and not expressing p53;
(g) making dynamic measurements using reporter molecules or genes of short half lives or that are excreted;
(h) determining or confining the targets of drug action in vivo;
(i) measuring drug bystander effects; and identifying promoter elements involved in gene regulation;
(j) determining intracellular drug concentrations and thereby those cells that take up a drug; and
(k) determining or confirming the targets of drug action in vivo.

According to a yet further aspect of the present invention there is provided a kit comprising at least one reporter cell/system as hereinbefore described and optionally a set of instructions therefore. It is envisaged that the kit of the present invention may be supplied as a suspension of reporter cells.

The invention will now be described by way of example only with reference to the following Figures wherein:

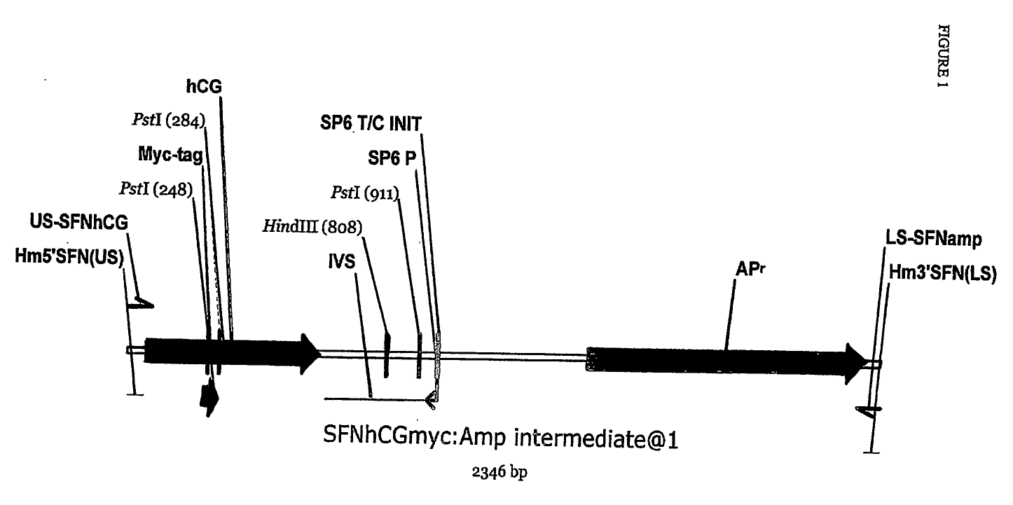

FIG. 1 shows the schematic generation of SFN-hCG(myc):Amp targeting construct;

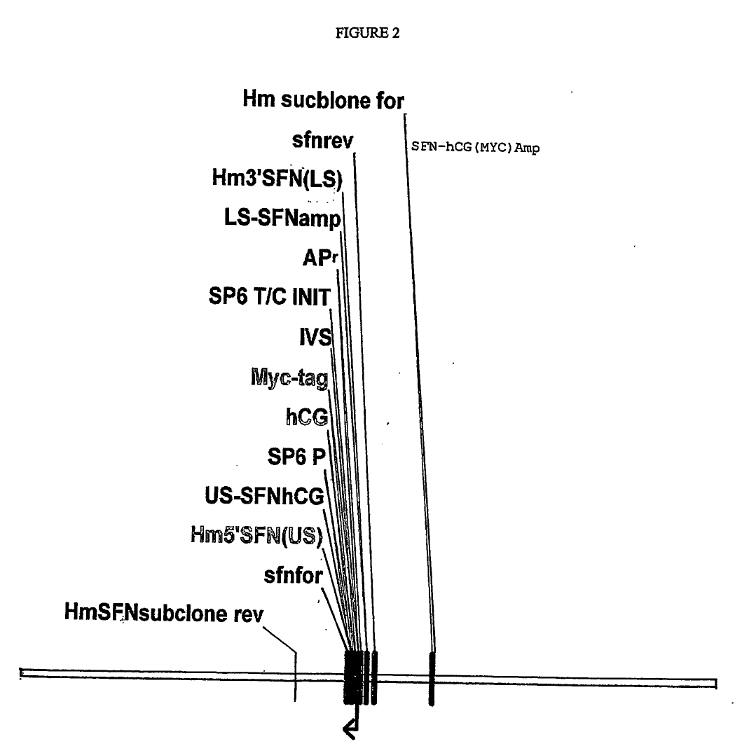

FIG. 2 shows SFN-hCG(myc):PAC clone;

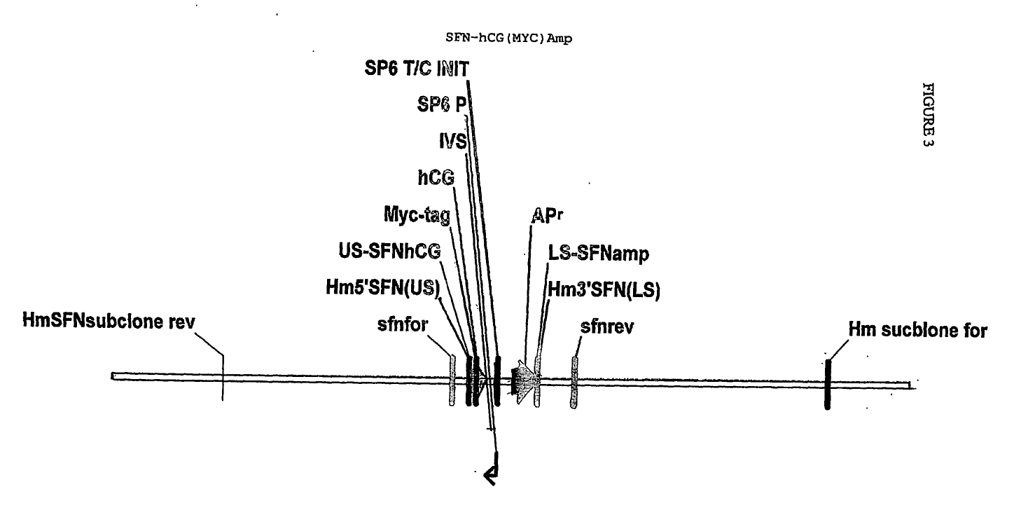

FIG. 3 shows detail of the restriction map of FIG. 2;

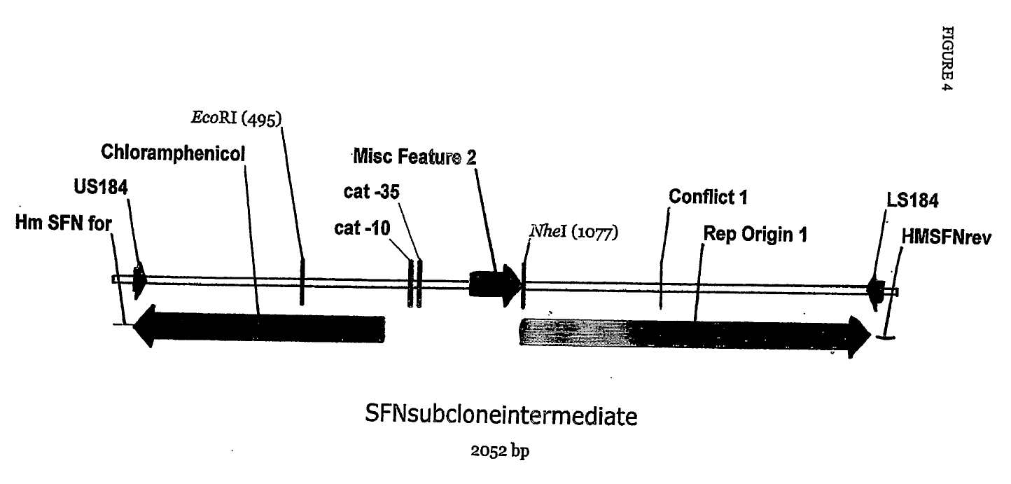

FIG. 4 shows the schematic generation of SFN subclone intermediate;

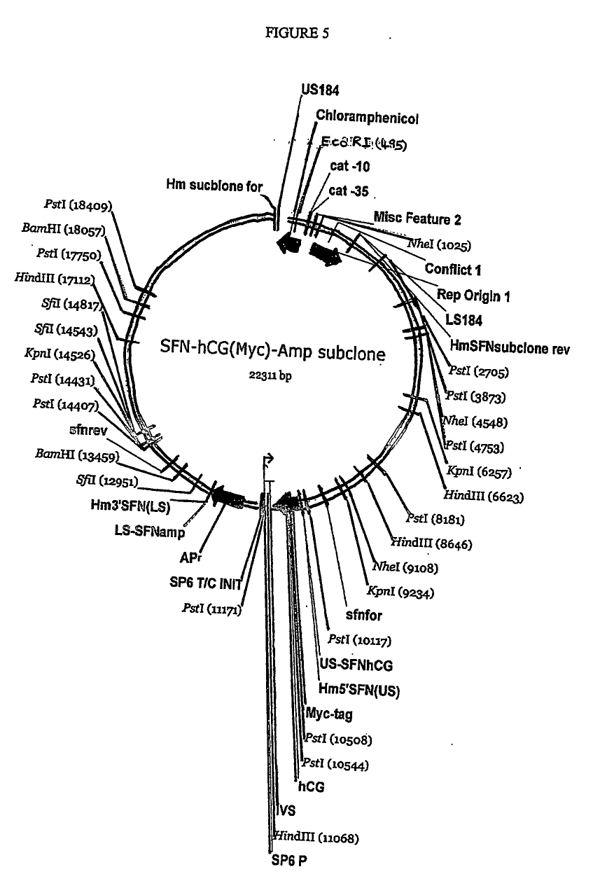

FIG. 5 shows the restriction map of the final construct;

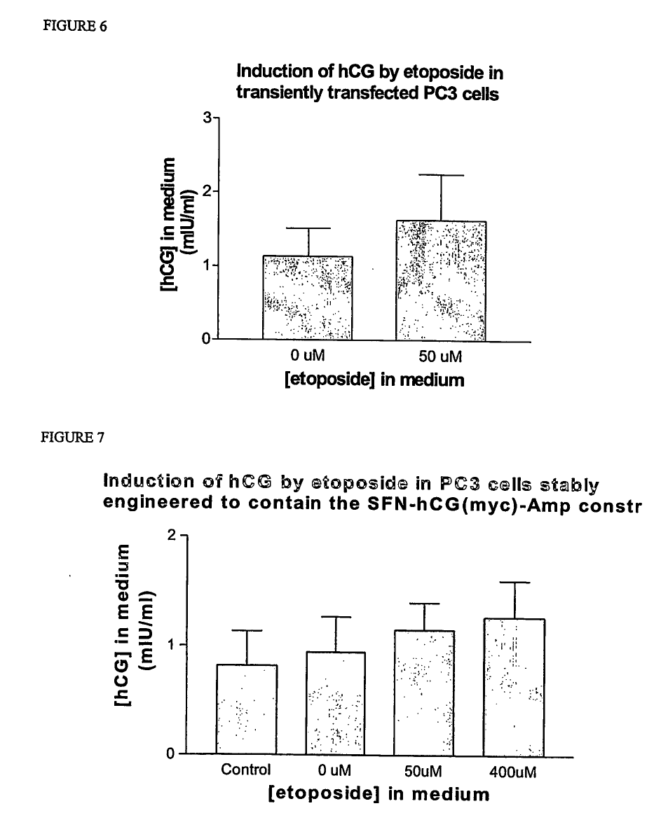

FIG. 6 shows induction of hCG by etoposide in transiently infected PC3 cells;

FIG. 7 shows induction of hCG by etoposide in PC3 cells stably engineered to contain the SFN-hCG(myc)-Amp construct;

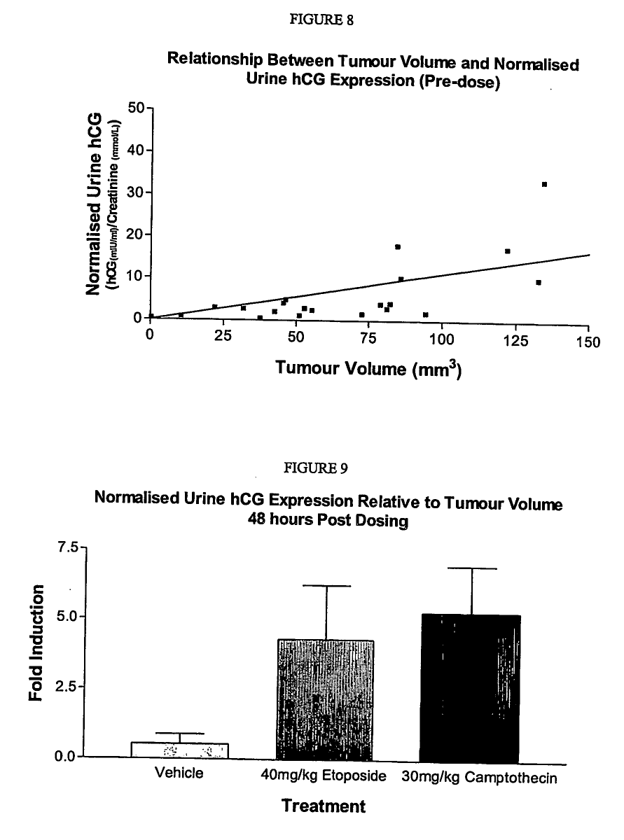

FIG. 8 shows a graph of the relationship between tumour volume and normalised urine hCG expression (pre-dose);

FIG. 9 shows normalised urine hCG expression relative to tumour volume 48 hours post-dosing;

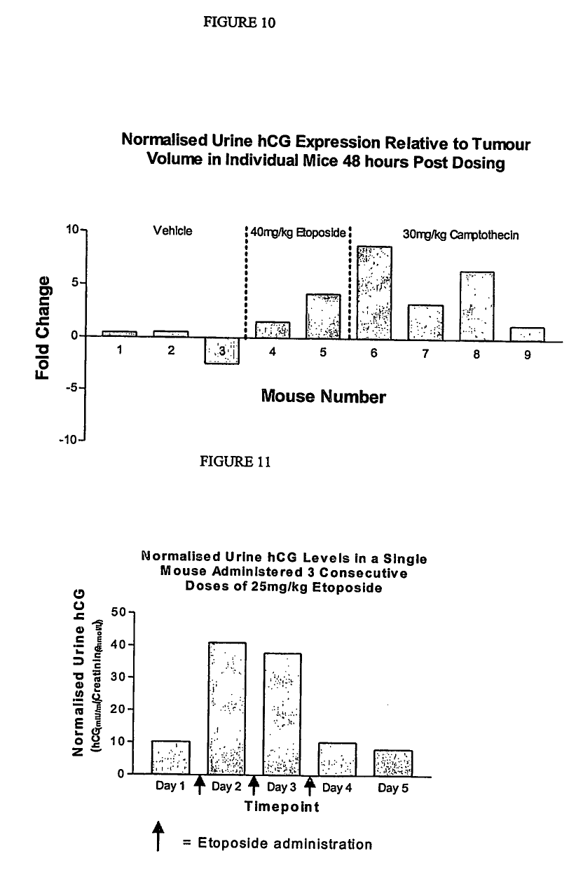

FIG. 10 shows normalised urine hCG expression relative to tumour volume 48 in individual mice hours post-dosing and;

FIG. 11 shows normalised urine hCG levels in a single mouse administered with 3 consecutive doses of 25 mg/kg etoposide.

### MATERIALS AND METHODS

Reporter Selection

The promoter of the Stratifin (SFN) gene (also known as 14-3-3σ), is a marker of G2/M arrest occurring as a result of DNA damage. The SFN gene has been shown to be transcriptionally upregulated via a p53-dependent mechanism during G2/M arrest in human tumour derived cell lines following γ-irradiation or treatment with adriamycin (also known as doxorubicin) [1]. Expression of SFN appears to be functionally involved in G2/M arrest in that its expression seems to halt progression through the G2/M checkpoint [1]. In addition, transcriptional activation of the SFN promoter can occur in response to the tumour suppressor protein BRCA1, whose transcriptional activation function is activated by DNA damage [2]. The facts that SFN induction precedes changes in p53 expression [3], and that BRCA1 expression is both necessary and sufficient for G2/M arrest and SFN induction in p53-deficient HCC1937 cells [4], indicate that this pathway of induction is p53-independent. Thus the induction of SFN by DNA damage appears to occur via both p53-dependent and p53-independent pathways [1, 3, 4].

An artificial gene construct in which the 5′-regulatory promoter region of the SFN gene was linked to the excretable reporter human chorionic gonadotrophin (hCG) was generated. A human tumour-derived cell line containing this construct was engineered and tested in vitro. This was then used in in vivo xenograft experiments to demonstrate a change in the excretion of hCG in response to treatment with anticancer drugs.

Generation of the SFN-hCG Construct

This reporter construct was assembled using recombination cloning utilising the Red/ET homologous recombination system (Genebridges).

The genomic clone of SEN (sfn PROTEIN} was identified using the Human Ensembl site, http://www.ensembl.org/Homo_sapiens, (supported by the Sanger Institute). A human PAC clone RPCI-50o24 was identified to contain the whole coding region and promoter and regulatory regions deemed essential for normal regulation. The PAC clone was further verified by PCR to contain both the 5′ and 3′ UTRs. The SFN oligos used for screening were:

SFN Verification of Oligos to Position 48,671-48,690 bp

| | | |
|---|---|---|
| SFN_for | ATG GTC CTG TGT GTG TCA C | (SEQ ID NO:1) |
| SFN_rev | CAG GGG AAC TTT ATT GAG A | (SEQ ID NO:2) |

Clones that gave the correct PCR product were then processed as follows. The verified PAC clones were transformed with the plasmid pSC101BADgbaA (Genebridges). This plasmid provides the recombinases essential for the recombination process. The PAC/pSC101BADgbaA clones were further verified for the presence of pSC101BADgbaA by restriction analysis. Only the clones that gave the correct restriction pattern were used.

The generation of hCG(myc):Amp targeting construct was undertaken as follows, however it will be appreciated that the construct does not necessarily have to include an epitope tag such as myc as our results indicate that the construct is functional in the absence of such a tag: The hCG(myc):Amp template had previously been cloned onto the equivalent of the pXEN backbone. This was digested to linearise the template to reduce background. The following oligonucleotides (BioSpring) were used to generate the targeting molecule:

US-SFNhCG

(SEQ ID NO:3)

TGGTCCCAGGCAGCAGTTAGCCCGCCGCCCGCCTGTGTGTCCCCAGAGCC
ATGGAGATGTTCCAGGGGCTG

LS-SFNamp

(SEQ ID NO:4)

TAGCGTTCGGCCTGCTCTGCCAGCTTGGCCTTCTGGATCAGACTGGCTCT
TTACCAATGCTTAATCAGTGA

The following reaction conditions were used: 39.5 ul dH2O, 5 ul 10× Tuning Buffer (Eppendorf), 2 ul 10 mM dNTPS (Roche), 1 ul US-SFNhCG (100 pmol), 1 ul LS-SFNamp (100 pmol), 0.5 ul Triple Master Taq polymerase (Eppendorf). PCR Block conditions (MWG): 94° C. 1 min×1 cycle, 93° C. 30 seconds, 56° C. 30 seconds×30 cycles, 72° C. 2 minutes 30 seconds, 72° C. 5 minutes×1 cycle. The molecule thus generated is shown in FIG. 1.

This PCRed targeting molecule was cleaned up (to further reduce background) by performing a Dpn1 digest on the total PCR reaction (this preferentially cuts methylated DNA, the template). The digested PCR reaction was ethanol precipitated and resuspended in water to give a final DNA concentration of 0.5 ug/ul. The pSC101ADgbaA containing PAC (RPCI-50o24) was cultured as follows; overnight 1 ml LB cultures (supplemented with Kanamycin 70 ug/ml and tetracycline 3 ug/ml) were grown at 30° C. with shaking at 1000 rpm. The next day three 1.4 ml Lb cultures, supplemented as previously, were set up, inoculated with 30 ul of the overnight culture and grown at 30° C. for 2 hours with shaking. After 2 hours two of the cultures were induced with 30 ul of L-arabinose (10%) and all three cultures were shifted to 37° C. with shaking for 1 additional hour (this induces the recombinases and stops the pSC101 BAD plasmid from further replication). The resulting cultures were then treated to make them electrocompetent by three washes in 1 ml if ice cold sterile water. The cells were then electroporated with the PCRed targeting molecule. After electroporation the cells were recovered for 70 minutes with 1 ml of LB broth at 37° C. The recovered cell were the plated out onto LB agar with the selection (Kanamycin 70 ug/ml and Ampicillin 20 ug/ml) and grown overnight at 37° C. The resulting colonies were screened for the correct recombination event for the generation of the modified PAC shown in FIG. 2, the detail of the restriction map region is seen in FIG. 3.

The screening regime was PCR across the junctions of hCG and SFN for the 5′ end and Amp and SFN for the 3′ end. On identification of positive clones the pSC101BADgbaA plasmid was re-introduced into the modified PAC and verified as previously described. The next stage was to subclone the modified SFN gene with 10 Kb of upstream sequence and some 9 Kb of downstream sequence onto a pACYC184 backbone. This was again achieved through the use of recombination cloning. The subcloning target construct was generated with PCR using the following oligos:

SFN Subcloning Oligos

SFN Subclone Forward

(SEQ ID NO:5)

TGCAGTGAGCCGAGATCTCGCCACTGCACTACTCCAGCCTGGGCGACAGA
GCTTACGCCCCGCCCTGCCACTC

SFN Subclone Reverse

(SEQ ID NO:6)

GGATATGGGAGCCAGCCACATTCATACAGGGCACACATGAACACACACAT
GTCAAACATGAGAATTACAA

PCR conditions were as previously described with the exception of the template used, linearised pACYC184. The PCR product was processed as previously described. The subcloning SFN target construct (SFN subclone intermediate) was generated as shown in the FIG. 4.

The modified SFN hCG(myc):Amp containing pSC101BADgbaA was made electrocompetent as previously described and electroporated with the SFN subclone intermediate. The resulting transformants were recovered in 1 ml of LB before being plated out onto LB agar plates supplemented with chloramphenicol 15 ug/ml and ampicillin 20 ug/ml. The potential transformants were screened by a number of diagnostic restriction digests and assessed by giving the correct restriction pattern. The clones giving the correct restriction pattern were bulk prepared by growing 400 ml liquid cultures (LB broth supplemented with chloramphenicol 15 ug/ml and ampicillin 20 ug/ml) and maxi prepped using the Qiagen Maxi kit (protocol followed contained within the kit). The restriction map of the final construct is shown in FIG. 5.

Generation of Cell Lines Containing the SFN-hCG(myc)-Amp Reporter Construct

The prostate tumour cell line PC3 is a p53^{-/-} cell line that can be grown as a monolayer in vitro and forms subcutaneous tumours when grown as a xenograft in congenitally athymic nude mice. Importantly, it has the capacity to undergo G2/M arrest following treatment with anticancer drugs [5].

The SFN-hCG(myc)-Amp reporter construct was transfected into PC3 cells on 6-well tissue culture plates using FuGene reagent (Roche, Lewes, East Sussex). Construct DNA (equivalent to 1 μg/well) was added to FuGene reagent (3 μl/well) and made up to 100 μl/well with serum free medium. The medium in which the cells were growing was aspirated off and replaced with 100 μl of the above mixture per well. For transient transfections, the cells were used for experiments 24 hours after the addition of the construct.

To generate a stable cell line, transfection was carried out as above. After 24 hours the cells were trypsinised and transferred to 10 cm tissue culture dishes (one well of a six well plate per 10 cm dish). The cells were allowed to grow on these dishes for 7 days prior to selection. After this time, G418 (20 ng/ml in cell culture medium) was added and the dishes maintained until colonies became visible (approximately one week). Individual colonies were picked using cloning discs soaked in typsin and transferred to individual wells of a 24 well plate. The colonies were then expanded to 6 well plates then T25 flasks and grown up until sufficient cells were present for use in in vitro inductions and xenograft experiments.

### EXAMPLE 1

In Vitro Exemplification

Etoposide is known to induce G2/M arrest via a BRCA1 linked mechanism in cultured cells [6].

Transiently transfected cells 24 hours after addition of the construct were treated with etoposide (50 μM or left untreated. After 24 hours in the presence or absence of 50 μM etoposide, samples of the medium were taken and assayed for hCG by enzyme-linked immunosorbent assay (Free Beta Human Chorionic Gonadotrophin Kit, Alpha Diagnostic International, Texas, USA). The results of this experiment are shown in the FIG. 6.

This demonstrates that the PC3 cells containing the SFN-hCG(myc)-Amp reporter construct secreted hCG into the surrounding medium and, furthermore, the amount secreted increased when the cells were treated with etoposide.

Following the generation of a stable cell line containing the SFN-hCG(myc)-Amp reporter construct, the cells were further tested using etoposide. The cells were plated out on 6 well plates and allowed to adhere overnight. The next day they were treated with etoposide (50 or 400 μM) or vehicle (dimethyl sulphoxide). After 24 hours in the presence or absence of etoposide, samples of the medium were taken and assayed for hCG by enzyme-linked immunosorbent assay as above. The results of this experiment are shown in FIG. 7.

This demonstrates that the PC3 cells stably engineered to contain the SFN-hCG(myc)-Amp contruct secreted hCG into the surrounding medium. As in the transient transfectants, the amount secreted increased when the cells were treated with etoposide. In addition, the amount of hCG secreted increased when the cells were exposed to a higher concentration of etoposide (400 μM vs 50 μMn.

### EXAMPLE 2

Xenograft In Vivo

PC3 cells stably transfected with the SFN-hCG(myc)-Amp reporter construct were allowed to grow as solid subcutaneous tumours in congenitally athymic nude mice. The mice were then treated with anticancer drugs that act by inducing G2/M arrest. The drugs chosen for this exemplification were etoposide and camptothecin [12].

For this experiment, wild-type PC3 cells (which do not express or secrete hCG) and the stable cell line containing the SFN-hCG(myc)-Amp reporter construct were used.

Wild-type and engineered PC3 tumour cell lines were cultured in RPMI medium supplemented with 10%-15% heat inactivated foetal calf serum, 2 mM L-glutamine, penicillin (50 IU/ml), streptomycin (50 μg/ml). Culture medium for PC3/SFN cells also contained G418 (200 μg/ml). Cultures were incubated in a humidified incubator at 37° C., 5% CO₂. Cells were harvested, pooled, centrifuged, and re-suspended in cold medium. This was mixed with an equal volume of cold Matrigel, so that the tumour cell injection solution was a 50:50 mixture of tumour cells/medium and Matrigel for each cell line. Wild type or transfected PC3 cells were injected at 2.5×10⁶ per animal. All cell lines were injected in a volume of 100 μl in the right hand flank only.

The study consisted of 4 groups in total, each containing 4 animals. One group of mice was implanted with wild type PC3 cells and the remaining 3 groups with engineered cells. Tumour growth was measured twice-weekly following cell implantation until tumours reached 2-5 mm in diameter. Tumour volume (V) was calculated using the formula: V= 4/3π((d1+d2)/4)³, where d=mean diameter (n=2)

Treatment began 5 weeks after tumour implantation. Wild type PC3-xenografted mice remained untreated; all other mice were administered vehicle only (DMSO/saline) or test substances. Single administrations of Camptothecin (30 mg/kg) and Etoposide (40 mg/kg) were administered ip at 10 ml/kg. Additionally, Etoposide (25 mg/kg) was administered to a single group of mice over three consecutive days (ip; 10 ml/kg). Urine samples were harvested before, during and after drug administration. Animals were sacrificed 48 hours after treatment. Urine was analysed for hCG and values normalised against urine creatinine levels.

No hCG was detected in the urine of mice injected with wild type PC3 cells, despite large tumour volumes (data not shown). Conversely hCG was detected in the urine of transfected PC3 cells, suggesting that the hCG detected here was expressed from the xenografted tumours. There was a positive correlation between tumour volume and urine hCG levels, as shown in FIG. 8. This demonstrates that there is a relationship between tumour volume and normalised urine hCG expression. The greater the tumour volume, the higher is the level of hCG expression.

Normalised hCG levels increased 0.54-, 4.29- and 5.29-fold when mice were administered single doses of vehicle only, 40 mg/kg etoposide or 30 mg/kg camptothecin respectively, as shown in FIG. 9.

Both etoposide and camptothecin significantly increased hCG expression in individual mice when compared to vehicle controls as shown in FIG. 10. This demonstrates that hCG expression in xenografted tumours is induced by the anti-cancer drugs etoposide and camptothecin as a result of increased DNA damage. This was shown by the increased levels of hCG that were detected in urine 48 hours after drug administration compared to pre-dose values.

Mice were administered 3 consecutive doses of etoposide at 25 mg/kg, and urine samples were harvested before, during and after drug administration. Following the first dose, there was a sharp increase in normalised hCG expression which remained at this level following a second dose of the test agent. Subsequent levels of hCG dropped to control levels at 24 and 48-hours following a third dose of etoposide. An example of this is shown in FIG. 11. The drop in hCG levels following administration of three consecutive doses of etoposide indicated that cell death, as a result of DNA damage, had occurred within the tumour. This suggested that etoposide exhibited an anti-tumour effect against the PC3/SFN cells; thereby reducing the number of hCG-expressing cancer cells, resulting in reduced hCG urine levels.

### REFERENCES

1. Hermeking, H., et al., (1997) 14-3-3 sigma is a p53-regulated inhibitor of G2/M progression. Molecular cell. 1: 3-11.
2. Somasundaram, K., (2003) Breast cancer gene 1 (BRCA1): Role in cell cycle regulation and DNA repair—Perhaps through transcription. Journal of Cellular Biochemistry. 88: 1084-1091.
3. Aprelikova, O., et al., (2001) BRCA1 is a Selective Co-activator of 14-3-3(sigma) Gene Transcription in Mouse Embryonic Stem Cells. Journal of Biological Chemistry. 276: 25647-25650.
4. Yarden, R. I., et al., (2002) BRCA1 regulates the G2/M checkpoint by activating Chlk1 inase upon DNA damage. Nature Genetics. 30: 285-289.
5. Aranha, O., et al., (2003) Suppression of human prostate cancer cell growth by ciprofloxacin is associated with cell cycle arrest and apoptosis. International journal of oncology. 22: 787-794.
6. Quinn, J. E., et al., (2003) BRCA1 functions as a differential modulator of chemotherapy-induced apoptosis. Cancer Research. 63: 6221-6228.
7. Siu, W. Y., T. Arooz, and R. Y. C. Poon, (1999) Differential responses of proliferating versus quiescent cells to adriamycin. Experimental Cell Research. 250: 131-141.
8. Siu, W. Y., C. H. Yam, and R. Y. C. Poon, (1999) G1 versus G2 cell cycle arrest after adriamycin-induced damage in mouse Swiss3T3 cells. FEBS Letters. 461: 299-305.
9. Su, J. and K. Ciftci, (2002) Changes in BRCA1 and BRCA2 expression produced by chemotherapeutic agents in human breast cancer cells. International Journal of Biochemistry and Cell Biology. 34: 950-957.
10. Goncalves, A., et al., (2000) Caspase-8 activation independent of CD95/CD95-L interaction during paclitaxel-induced apoptosis in human colon cancer cells (H29-D4). Biochemical Pharmacology. 60: 1579-1584.
11. Vikhanskaya, F., et al., (1998) Inactivation of p53 in a human ovarian cancer cell line increases the sensitivity to paclitaxel by inducing G2/M arrest and apoptosis. Experimental Cell Research. 241: 96-101.
12. McDonald, A. C. and R. Brown, (1998) Induction of p53-dependent and p53-independent cellular responses by topoisomerase 1 inhibitors. British Journal of Cancer. 78: 745-751.

1. A method of monitoring progression of a xenograft in a non-human host animal comprising:
(i) genetically modifying or engineering a cell before or after implantation into an animal so as to incorporate at least one reporter molecule and/or reporter gene and/or reporter agent into said cell;
(ii) implanting said modified cell into said host animal and allowing a xenograft to grow for a sufficient period of time; and
(iii) measuring at least one parameter of a selected biochemical/physiological response associated with the reporter molecule or reporter gene.

2. The method according to claim 1 wherein there is a plurality of genetically modified or engineered cells which are human or non-human in origin.

3. The method according to claim 1 wherein the cell is a primary isolate derived from normal tissue or a tumour or is an immortalized or established cell line.

4. The method according to claim 1 wherein the reporter molecule is selected from the group consisting of a protein, an antigen, an enzyme, an enzyme substrate, a fluorescent agent, a chemiluminescent agent, a chromogenic agent and a radionuclide.

5. The method according to claim 1 wherein the reporter gene is selected from those genes encoding proteins chloramphenicol-acetyltransferase, \tilde{β}-galactosidase, \tilde{β}-glucuronidase, luciferase, beta-galactosidase or green fluorescent protein, secreted alkaline phosphatase (SEAP), major urinary protein (MUP) or human chorionic gonadotrophin (hCG).

6. The method according to claim 1 where the reporter agent is a protease or kinase or the reporter is a protein or RNA effecting changes in protein or mRNA stabilization.

7. The method according to claim 1 wherein the host animal is a rodent.

8. The method according to claim 7 wherein the rodent is a mouse or rat.

9. The method according to claim 7 wherein the rodent is a wild type or genetically engineered mouse or rat having a specifically selected genetic background.

10. The method according to claim 1 wherein the host animal has more than one different population of reporter cells/system implanted therein.

11. The method according to claim 1 wherein the step comprising measuring at least one parameter of a selected biochemical/physiological response associated with the reporter molecule, reporter gene or reporter agent is a qualitative or quantitative measurement and optionally comprises invasive or non-invasive procedures for making the measurement.

12. The method according to claim 1 wherein the xenograft proliferates as a xenograft tumor with or without metastatic tumors at secondary sites.

13. The method according to claim 1 wherein the implanted modified cells are introduced into the host animal either as individual cells suspended in a suitable medium or as tumor fragments.

14. The method according to claim 1 wherein the implanted modified cells to grow in the host animal either systemically or as a xenograft tumor at the site of implantation.

15. The method according to claim 1 wherein the host animal is immunosuppressed by a method comprising administration of appropriate immunosuppressant agents, (ii) an immunocompromised strain or (iii) immunologically intact and wherein the implanted modified cell is synergistic with the host animal.

16. The method according to claim 1 wherein the reporter cell/system is genetically engineered to express a transgene or multiple transgenes.

17. The method according to claim 1 wherein the reporter cell/system expresses the reporter gene(s) or agent(s) at the time of implantation or is transfected in vivo with the reporter gene or agent in a specifically targeted manner.

18. The method according to claim 1 wherein the reporter gene(s) or agent(s) comprise at least one element that allows measurement of a biochemical parameter in response to either changes in cell physiology occurring during reporter cell/system proliferation or as a result of toxicological or pharmacological effects of an administered xenobiotic-compound or biological substance.

19. The method according to claim 1 wherein the step of measuring at least one parameter of a selected biochemical/physiological response associated with the reporter molecule, reporter gene or reporter agent comprises measuring or monitoring any one or more of the following parameters:
(a) reporter cell numbers, cell cycle modulation or mitotic fraction, cell differentiation, angiogenesis, hypoxia, cell death by necrosis, cell lysis or apoptosis;
(b) oxidative stress, DNA damage, mitochondrial function, membrane perturbation, GSH depletion, receptor-mediated toxicity, enzyme inhibition, cofactor availability, pH or osmotic change, perturbation of calcium homeostasis, cell differentiation, protein turnover, ubiquitination or protein misfolding;
(c) effects on intracellular signalling pathways, receptor interactions, effects on gene transcription, translation or protein stability, hormone or growth factor receptor modulation, peroxisome proliferator-activated receptor modulation, intracellular signal transduction pathways, MAP kinase or phosphatase signalling, p53 signalling or ras signalling; or
(d) induction of drug resistance mechanisms, drug delivery or drug bystander effects.

20. The method according to claim 1 wherein the reporter gene comprises a naturally occurring or artificial promoter sequence driving expression of a gene resulting in production of a reporter protein.

21. The method according to claim 20 wherein the promoter is constitutively active or is inducible.

22. The method according to claim 1 wherein the reporter gene expression product is reportable transcriptionally or post-transcriptionally.

23. The method according to claim 22 wherein transcriptional reporting is mediated by any one of the gene promoters selected from the group consisting of vascular endothelial growth factor (VEGF), nitric oxide synthetase (iNOS) promoter, haemoxygenase-1 (HO-1) promoter, cyclo-oxygenase-2 (COX-2) promoter, transglutaminase promoter, Peg3/pwl promoter, 14-3-3 protein promoter and a GADD153 promoter.

24. The method according to claim 22 wherein post-transcriptional reporting is mediated through generation of a protein that can effect protein modifications selected from the group consisting of a consequence of protease activity that results in translocation of a cytoplasmic protein to the nucleus or from membrane-bound form to secreted form, protein cleavage, activation of a proenzyme or transcription factor, deactivation of an active enzyme or transcription factor and secretion into the blood or excretion into urine.

25. The method according to claim 22 wherein post-transcriptional reporting is mediated through production of a protein or RNA that effects changes in the stabilization of a protein or mRNA.

26. The method according to claim 22 wherein post-transcriptional reporting is mediated through production of a protein which on death or lysis of the cell expressing it.

27. The method according to claim 1 wherein measuring of at least one parameter of a selected biochemical/physiological response associated with the reporter molecule or reporter gene or reporter agent comprises a non-invasive or an invasive assay wherein:
(i) the non-invasive assay is in excreted body products, or by bioluminescence measurement, or by blood pressure measurement, or by transcutaneous oxygen tension measurement, or by nuclear magnetic resonance measurement or by positron emission tomographic measurement; or
(ii) an invasive assay for blood or xenograft reporter products.

28. The product produced by the method of claim 1 and comprising a genetically modified or engineered cell, the modification or engineering of the cell being such that the cell comprises at least one reporter molecule or reporter gene.

29. (canceled)

30. An artificial gene construct comprising a promoter region of the SFN gene and human chorionic gonadotrophin (hCG) such that expression of hCG is controlled by the SFN promoter.

31. A construct according to claim 30 wherein 5′-regulatory promoter region of the SFN gene is linked to hCG.

32. The human tumor-derived cell line comprising the construct of claim 30.

33. The non-human animal comprising a human tumor-derived cell line according to claim 32.

34. The method of claim 1 further comprising:
(a) measuring reporter cell proliferation, in response to treatments;
(b) determining the mechanism of differentiation or death where reporter cell differentiation or death occurs;
(c) monitoring processes in secondary metastatic tumors;
(d) making non-invasive measurements of parameters related to biochemical processes in the reporter cell/system;
(e) identifying drug-resistant cell populations;
(f) determining the effects of genetic background on tumor cell growth or on response to treatments;
(g) making dynamic measurements using reporter molecules or genes of short half lives or that are excreted;
(h) determining or confirming the targets of drug action in vivo;
(i) measuring drug bystander effects; and identifying promoter elements involved in gene regulation;
(j) determining intracellular drug concentrations and thereby those cells that take up a drug; and
(k) determining or confirming the targets of drug action in vivo.

35. A kit comprising a product with at least one reporter cell/system of claim 28 and optionally a set of instructions therefor.

36. The kit according to claim 35 wherein the product is supplied as a suspension of reporter cells.
